(19) United States
(12) Patent Application Publication
(10) Pub. No.: US 2017/0151134 A1
(43) Pub. Date: Jun. 1, 2017

(54) Oil-Enclosed Powdery Composition and Production Method of the Same

(71) Applicant: SHISEIDO COMPANY, LTD., TOKYO (JP)

(72) Inventors: Motoharu KIMURA, KANAGAWA (JP); Takuya HIRUMA, KANAGAWA (JP); Tetsuya KANEMARU, KANAGAWA (JP); Masayuki SHIRAO, KANAGAWA (JP); Tomoko SATO, KANAGAWA (JP); Toru YU, Shizuoka (JP)

(73) Assignee: SHISEIDO COMPANY, LTD., TOKYO (JP)

(21) Appl. No.: 15/110,908

(22) PCT Filed: Feb. 24, 2016
(86) PCT No.: PCT/JP2016/055407

§ 371 Date: Jul. 11, 2016

(30) Foreign Application Priority Data
Jul. 16, 2015 (JP) 2015-142165

(51) Int. Cl. A61K 8/11; A61K 8/55; A61K 8/26; A61K 8/25; A61Q 1/02; A61K 8/31

(52) U.S. Cl. CPC A61K 8/11; A61Q 1/02; A61K 8/31; A61K 8/26; A61K 8/25; A61K 8/55; A61K 2800/63; A61K 2800/412; A61K 2800/43

(57) ABSTRACT

An oil-enclosed powdery composition includes an oil-enclosable powder; further comprising; a platy powder, aluminum hydroxide coating the platy powder, having a surface of a specific surface area of at least 10 m²/g, and a fluorine compound coating the aluminum hydroxide; and an oil droplet having at least 50% by mass of hydrocarbon oil based on a total oil component and at least 2.0×10⁻² N/m of a surface tension value as a total oil component; wherein the oil-enclosable powder encompassing the oil droplet is like powder having an average particle size of 10 to 150 μm and the oil-enclosed powdery composition is liquefiable by inunction.

### RELATED APPLICATIONS

The present invention claims priority from Ser. No. PCT/JP2016/055407 filed Feb. 24, 2016, the contents of which are incorporated by reference which is based on Japanese Patent Application No. 2015-142165 filed on July 2015, the disclosure of the application is incorporated herein in its entirety by reference.

The present invention claims the priority based on Japanese Patent Application: No. 2015-142165 (filed on Jul. 16, 2015), the disclosure of the application is incorporated herein in its entirety by reference.

### TECHNICAL FIELD

The present invention relates to an oil-enclosed powdery composition and a production method thereof, and particularly, relates to the improvement of the surface condition of the oil-enclosable powder.

### BACKGROUND ART

An oil-enclosed powdery composition having an powdery appearance as if a powdery composition, in which oil droplets comprising the oil component that is encompassed by oil-enclosable powder, is disclosed, wherein the powdery composition provides a paste or an oily property by inunction (Patent literature 1).

Such powdery composition; in which the oil component having a high surface tension, as-is oil droplets, is encompassed by the fluorine-treated powder having superior water repellency and oil repellency, is stable because of the high surface tension of the oil droplets; further can maintain the condition under which the oil-enclosable powder is encompassing the oil droplets based on “weak wettability” of the oil-enclosable powder; and further may be called as a dry oil because such powder disperses inside the oil and turns to paste or a liquid form by inunction.

### CITATION LIST

### Patent Literatures

[Patent literature 1] Japanese Patent Publication No. 3812733

### SUMMARY OF THE INVENTION

### Technical Problem to be Solved

The conventional oil-enclosed powdery composition may turn to paste or liquid during manufacturing or transportation of a product. The present inventors have continued the investigation and have found that a proper oil-enclosed condition may be unlikely kept when the oil-enclosed powdery composition is left under vibration.

The present invention was made in view of the above-described conventional art, and the problem to be solved is to provide an oil-enclosed powdery composition that can provide an excellent production applicability and can have high stability.

### Means to Solve the Problem

The present inventors have diligently studied to solve the above-described problem and as a result, have found that the properties such as the stability of the oil-enclosed powdery composition drastically vary depending upon the surface condition of the fluorine-treated powder, the kinds of oil to be enclosed, and the size of enclosed oil droplets, and then completed the present invention.

Specifically, an oil-enclosed powdery composition according to the present invention comprises: an oil-enclosable powder; further comprising; a platy powder, aluminum hydroxide coating the platy powder, having a surface of a specific surface area of at least 10 m²/g, and a fluorine compound coating the aluminum hydroxide; and an oil droplet having at least 50% by mass of hydrocarbon oil based on a total oil component and at least 2.0×10⁻² N/m of a surface tension value as a total oil component; wherein the oil-enclosable powder encompassing the oil droplet is like powder having an average particle size of 10 to 150 μm and the oil-enclosed powdery composition is liquefiable by inunction.

In addition, relative to the composition, it is preferred that the platy powder is sericite.

In addition, relative to the composition, it is preferred that the ratio of the oil-enclosable powder vs. the oil component is 1:9 to 4:1 (mass ratio).

A production method of an oil-enclosed powdery composition according to the present invention comprises; the steps of: providing an oil-enclosable powder comprising a platy powder; aluminum hydroxide coating the platy powder, having a surface of a specific surface area of at least 10 m²/g; and a fluorine compound coating the aluminum hydroxide; stirring the oil-enclosable powder while spraying with a spray containing oil droplets having at least 50% by mass of hydrocarbon oil based on a total oil component and at least 2.0×10⁻² N/m of a surface tension value as a total oil component; and encompassing the oil droplets with the oil-enclosable powder during the step of stirring.

### Advantageous Effects of the Invention

According to the oil-enclosed powdery composition of the present invention, an oil-enclosed powdery composition providing an excellent production applicability and having high stability can be obtained by preparing an oil-enclosed powdery composition using the oil-enclosable powder, wherein aluminum hydroxide precipitates on the surface of a platy powder to provide the specific surface area of not smaller than 10 m²/g, and it is further coated with a fluorine compound; and an oil component wherein hydrocarbon oil is not less than 50% by mass based on the total oil component, the total surface tension value of the oil component is not smaller than 2.0×10⁻² N/m, and the average particle size of oil droplets is 20 to 80 μm.

### BRIEF DESCRIPTION OF THE DRAWINGS

FIG. 1 illustrates the appearance of an oil-enclosed powdery composition according to one embodiment of the present invention.

FIG. 2 illustrates the appearance of the oil-enclosed powdery composition of FIG. 1 after a vibration test.

FIG. 3 illustrates the appearance of the oil-enclosed powdery composition according to a comparative example to the present invention.

FIG. 4 illustrates the appearance of the oil-enclosed powdery composition of FIG. 3 after a vibration test.

FIG. 5 illustrates the magnified view of the appearance of oil-enclosed powder of the present invention.

### MODES

### Oil-Enclosable Powder

An oil-enclosable powder suitably used in the present invention is a powder that encompasses oil droplets described below, and it is preferably a fluorine-treated aluminum-hydroxide-coated platy powder. Here, the fluorine-treated aluminum-hydroxide-coated platy powder is formed by precipitating aluminum hydroxide on the surface of a platy powder including such as sericite, mica, etc., and generating water repellency and oil repellency by further adding a fluorine group.

### [Powder to be Treated]

As the powder to be treated, platy powder is preferably used. Examples of the platy powder may include sericite, mica, talc, alumina, silica, platy barium sulfate, bismuth oxychloride, boron nitride, etc. and further preferably platy clay minerals can be used therefrom. As the platy clay mineral, either natural or synthetic types can be arbitrarily used. As such clay mineral, sericite (microcrystalline hydrous aluminum potassium silicate; K₂O.3Al₂O₃.6SiO₂, 2H₂O), mica (hydrous aluminum potassium silicate; KAl₂.AlSi₃O₁₀(OH)₂), talc, etc. can be preferably used. By using such clay minerals, the later-described oil components can be blended in a higher amount while a powdery form is being maintained, and a further improvement in the usability (no frictional feeling, no rough skin feeling, etc.) can be achieved.

As to the powder component to be treated, for example, a composite pigment such as titanium oxide sericite, on which the clay mineral is coated with titanium oxide, can also be used.

As to the form of the powder to be treated, platy powder can be preferably used because oil can be effectively made powdery with a small amount of powder.

[Coating with Aluminum Hydroxide]

In the present invention, the precipitation of aluminum hydroxide is carried out before the above-described fluorine treatment. Thus, the coating with a fluorine compound can be stably and effectively processed by fluorine treatment following the precipitation of aluminum hydroxide, and a fluorine-treated powder having superior water repellency and oil repellency can be produced. In addition, when the powder fluorinated following coating with aluminum hydroxide is used, the impact stability (impact resistance) can be significantly enhanced. In addition, a superior skin adhesion effect is generated.

In the precipitation of aluminum hydroxide on the powder surface, for example, water is added to the powder to make a slurry, an aqueous solution of the above-described strongly-ionic water-soluble aluminum compound such as aluminum chloride or sodium aluminate is added to such slurry, and the compound is adsorbed on the powder surface. Subsequently, an acid or alkaline solution is added, the hydrolysis or substitution reaction of the compound adsorbed on the powder surface is carried out and the above-described metal hydroxide or the hydrate, partial dehydrate, or anhydride of the metal salt is formed.

The smaller amount of the above-described metal hydroxide or the hydrate, partial dehydrate, or anhydride of the metal salt that coats the powder is the more preferable and the amount of metal compound used for the production thereof is preferably 1% to 30% by mass relative to the powder. If the amount is less than 1% by mass, it is difficult that the effect of metal treatment can be satisfactorily achieved and on the other hand, if the amount exceeds 30% by mass, the primary function of the powder tends to be disturbed because of being bulky.

### [Fluorine Treatment]

Subsequently, an emulsion formed by adding water to a fluorine compound is gradually added to the surface metal-treated powder above-described and then the powder coated with the above-described metal hydroxide or the hydrate, partial dehydrate, or anhydride of the metal salt can be further coated with the fluorine compound by means of breaking the emulsion by acid or standing at high-temperature.

The molecule having the above-described fluorine group is not limited in particular and the examples may include fluorine compounds such as perfluoroalkyl phosphoric acid ester diethanolamine salt represented by the following general formula (I), perfluoroalkylsilane represented by the following general formula (II), and perfluoroalkylethyl acrylate represented by the following general formula (III).

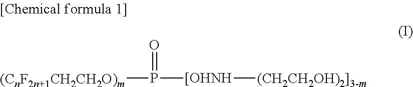

(In the formula, n represents an integer of 5 to 20 and m represents 1 or 2.)

[Chemical formula 2]

CₐF₂ₐ₊₁—(CH₂)_{b}—SiX₃ (II)

(In the formula, a represents an integer of 1 to 12, b represents an integer of 1 to 5, and X represents a halogen atom, alkyl group or alkoxy group.)

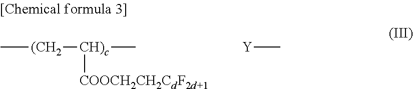

(In the formula, c represents an integer of 1 to 12, d represents an integer of 5 to 20, and Y represents polyethylene glycol, a silicone chain or an alkyl copolymer containing acryl groups etc.)

In addition, fluorine compounds having a perfluoropolyether group represented by perfluoropolyether dialkylphosphoric acid and salts thereof, perfluoropolyether dialkylsulfuric acid and salts thereof, and perfluoropolyether dialkylcarboxylic acid and salts thereof can be used. Here, the “perfluoropolyether group” means a group having at least two or more oxygen atoms that are bonded to perfluoroalkylene or perfluoroalkyl, and the one with the molecular weight of about 300 to 7000 is preferable from the standpoint of oil repellency and water repellency.

The method of fluorine treatment is not limited in particular; for example, the following method can be included. Specifically, a fluorine-treated powder can be obtained by gradually adding a fluorine compound aqueous solution to an aqueous dispersion of the powder to be treated, adjusting the solution to acidic condition by the addition of hydrochloric acid aqueous solution, ageing for about 2 hours, filtering, drying and powdering.

In addition, so far as the surface is treated to provide water repellency and oil repellency by adding a fluorine group on the powder surface, any other non-fluorine group including such as an alkyl group, a silicone group or a hydrophilic group can be further added within the range that does not impair the effect of the present invention.

The fluorine compound used in the fluorine treatment is preferably 1% to 30% by mass with respect to the powder. If it is less than 1% by mass, the coat formation with the fluorine compound is unsatisfactory so that it can be difficult to realize water repellency and oil repellency and on the other hand, if it exceeds 30% by mass, it becomes bulky so that the primary function of the powder tends to be disturbed.

Further, relative to the powdery composition of the present invention, in addition to the above-described oil-enclosing powder, a powder without fluorine treatment and a pharmaceutical agent may be as-is blended as a powder component within the range that does not impair the effect of the present invention.

### [Oil Component]

While the oil component comprises one or more oil components, the (average) surface tension value thereof is not lower than 2.0×10⁻² N/m (relative to atmosphere) and preferably not lower than 2.1×10⁻² N/m. If the surface tension value of the oil component is lower than 2.0×10⁻² N/m, the oil component easily wets the powder so that the oil component cannot be in a good powdery form.

“The surface tension value of an oil component” is the surface tension value of the total oil component that forms a liquid. So far as the surface tension value of the total oil component is satisfactory in the above-described range, any of each oil component having a surface tension value lower than 2.0×10⁻² N/m can be applied as a constitutional component of the oil component.

Accordingly, the oil component may include any type of oil component, including such as solid, semi-solid and liquid and so forth. In addition, such oil component may be included in not only a homogeneous solution system but also a dispersion system in which oil is dispersed in another oil.

As such oil component, for example, hydrocarbon oils such as liquid paraffin, ozokerite, squalene, pristane, paraffin, ceresin, squalane, and petrolatum and so forth may be preferably used and especially preferably, the percentage of hydrocarbon oil in the oil component is not less than 50% by mass. As the oil component other than hydrocarbon oil, polar oils such as liquid lanolin, isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, acetylated lanolin, isocetyl stearate, isocetyl isostearate, cholesteryl 12-hydroxystearate, ethylene glycol di-2-ethylhexanoate, dipentaerythritol fatty acid ester, N-alkylglycol monoisostearate, neopentyl glycol dicaprate, diisostearyl malate, glyceryl di-2-heptylundecanoate, trimethylolpropane tri-2-ethylhexanoate, trimethylolpropane triisostearate, pentaerythritol tetra-2-ethylhexanoate, glyceryl tri-2-ethylhexanoate, trimethylolpropane triisostearate, cetyl 2-ethylhexanoate, 2-ethylhexyl palmitate, glyceryl trimyristate, glyceride tri-2-heptylundecanoate, castor oil fatty acid methyl ester, oleic acid, acetoglyceride, 2-heptylundecyl palmitate, diisopropyl adipate, N-lauroyl-L-glutamic acid 2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, ethyl acetate, butyl acetate, amyl acetate, and triethyl citrate; higher alcohols such as stearyl alcohol, behenyl alcohol, oleyl alcohol, and cetostearyl alcohol; and plant oils such as jojoba oil, olive oil, nut oil, safflower oil, and soybean oil; can be included.

Silicone oils including; such as, a linear silicone having a low surface tension, including such as dimethylpolysiloxane, methylphenylpolysiloxane, methylhydrogenpolysiloxane and so forth and a cyclic silicone including such as octamethylcyclotetrasiloxane, decamethylcyclopentasiloxane, dodecamethylcyclohexasiloxane and so forth, and fluorocarbon oils as represented by “Fomblin®” series (manufactured by Ausimont) can also be used, if the amount is small and within the range in which the (average) surface tension value of the total oil component is not lower than 2.0×10⁻² N/m. When a large amount of such a silicone oil or fluorocarbon oil, which has a small surface tension value, is used, the surface tension value is too low to satisfactorily make the oil component powdery, in case. The surface tension value can be measured by using a publicly known method such as the pendant drop method.

In addition, semi-solid oil components such as hydrogenated palm oil, hydrogenated castor oil, and petrolatum; and solid oils such as paraffin wax, microcrystalline wax, carnauba wax, and candelilla wax can also be used within the range that the total surface tension value of the oil component satisfies the above-described range.

Especially in the case of powdery cosmetics, the preferred oil component is an oil component having the melting point of about 37° C. or lower so that melting can take place on the skin, but solid oils such as waxes can be blended so far as the amount is small.

Within the range, in which the effect of the present invention is not impaired, oil-soluble vitamins such as vitamin A and their derivatives, sterols, natural and synthetic perfumes, UV absorbers and poorly water-soluble materials, coloring materials, fine powders, etc. may be blended in the oil component.

### [Preparation of Oil-Enclosed Powdery Composition]

It is preferred to prepare the oil-enclosed powdery composition of the present invention by adding the oil component as oil droplets to the oil-enclosable powder and mixing under stirring.

In the present invention, the blending quantity of the oil-enclosable powder is preferably about 10% to 80% by mass based on the total composition, and more preferably about 20% to 70% by mass. If the blending quantity is too small, the oil component can be insufficiently powdery and accordingly, the intended powdery form may not be obtained. On the other hand, if the blending quantity is too much, liquefying thereof by inunction during use can be hard and not preferred from sensory standpoints.

When a powder component other than the oil-enclosable powder is contained, the blending percentage of the oil-enclosable powder with respect to the total powder component is preferably 50% to 100% by mass.

According to the present invention, the blending quantity of the oil component can be up to the balance of the total blending quantity of other essential components and optional additive components in the composition, but it is preferably 20% to 90% by mass. If the blending quantity of the oil component is too small, liquefying thereof by inunction during use can be hard and not preferred from sensory standpoints. On the other hand, if the blending quantity is too large, it is difficult to make powdery therefor. According to the present invention, especially when a clay mineral is used as the powder component of the oil-enclosable powder, the oil component can be blended in a high amount while a powdery form is being maintained. Especially in the case of powdery cosmetics, the excellent usability could be obtained based on suppressing frictional feeling, sticky feeling and rough feeling.

According to the present invention, it is preferred that the ratio of the oil-enclosable powder vs. the oil component is 1:9 to 4:1 (mass ratio) and more preferably 1:5 to 3:1.

In the powdery composition of the present invention, especially in the case of powdery cosmetics, any variety of optional components used in normal cosmetics, for examples, including perfumes, various powders, oil-soluble pharmaceutical components and so forth, can be blended within the range, in which the effect of the present invention is not impaired, in addition to the above-described components.

The powdery composition of the present invention may be practically contain no water, and, in particular, when the powdery composition is used for the powdery cosmetic, the formulation without blending additive components such as preservatives is affordable and the product that meets recent consumer preference for safety and security relative to the cosmetic industry can be commercialized.

In addition, the powdery composition can be blended in the formulation with no surfactant according to the present invention. However, a surfactant can be suitably blended depending on the intended use of cosmetics such as a powdery makeup remover for local use. The powdery makeup remover in which a surfactant is blended as an additive component provides a high makeup removal effect without dripping so that the usability can be improved and further rinsing-off with water can be provided. When a surfactant is used in the powdery makeup remover for local use, the blending quantity of the surfactant is preferably about 0.1% to 40% by mass relative to the oil component. When a surfactant is blended, it is preferred that the surfactant should be blended in the oil component with other oils. As such surfactant, any nonionic surfactants, any anionic surfactants and any cationic surfactants can be blended. Such surfactants can be any kind, e.g., either hydrophilic or lipophilic. The surfactant that is not soluble in the oil phase can also be blended by dispersing in the oil phase as the form of emulsion or solid powder.

In the powdery composition according to the present invention the oil-enclosable powder adsorbs to the periphery of the oil component and the oil component is turned powdery by the oil-enclosable powder so that the usage texture and the effectiveness of the oil component can be achieved by that such adsorption condition is disturbed and the oil component turned powdery oozes out therefrom and liquefies when a force is applied by inunction.

A production method of the powdery composition of the present invention is not limited in particular so far as the oil-enclosable powder adsorbs to the periphery of the oil component and the oil component is turned powdery by the oil-enclosable powder. For example, the production method wherein the oil-enclosable powder is added and mixed with an oil component or the oil component in which optional additive components are additionally dissolved can be included, but the production method is not limited to such examples.

When the composition of the present invention is applied to a powdery paint, for example, by using an oil dye as the oil component and uniformly coating the oil dye with fluorine-treated powder, there is a merit in that painting can be easily done on the wall, paper, etc., just by applying the paint having the powdery form with a brush.

### Examples

Hereinafter, the present invention is further explained with reference to examples; however, the present invention is not limited to the below-described examples. All the blending quantities are expressed in % by mass.

Ahead of the examples, test methods and evaluation methods used in the present invention are explained.

### [Dry Oil Formation: Production]

A: Stable dry oil can be prepared.
B: Dry oil can be prepared, but the paste is partially present.
C: Half or more is in a slurry form.
D: It is in a slurry form and dry oil cannot be prepared.

### [Impact Resistance]

Each example product was filled up to the mouth of 20 mL glass sample tube and the condition of the inside was visually observed following 10 times dropping on a rubber plate from the height of 70 cm and the result was evaluated based on the following criteria.

A: No change (a powder form was maintained.)
B: Pasty material adhered to a part of the inside of the tube, but overall nothing is problematic.
C: Adhesion of pasty material was noticeable.
D: The whole was pasty.

### [Vibration Test]

10 g of prepared dry oil was placed in a container and vibrated for 30 minutes.

A: No change (a powder form was maintained.)
B: Pasty material adhered to a part of the inside of the tube, but overall nothing is problematic.
C: Adhesion of pasty material was noticeable.
D: The whole was pasty.

### [Amount of the Enclosed Oil]

The added amount of the oil by the time immediately before a slurry was appeared when the oil was gradually added to the test tube containing 1 g of powder.

### [Surface Tension Value of Oil Component(s)]

The measurement was carried out by using the pendant drop method.

### [Specific Surface Area]

The measurement was carried out by using the BET (Brunauer-Emmett-Teller) method.

At first, the present inventors investigated the oil-enclosable powder that is the characteristically required element for the oil-enclosed powdery composition.

The results are shown in Table 1.

TABLE 1

Test Examples

| | 1-1 | 1-2 | 1-3 | 1-4 |
|---|---|---|---|---|
| Sericite | 50 | — | — | — |
| Fluorine-treated Sericite | — | 50 | — | — |
| Fluorine-treated and | — | — | 50 | 50 |
| Aluminum-hydroxide-coated Sericite | | | | |
| Liquid Paraffin | 50 | 50 | 50 | — |
| Dimethyl Silicone | — | — | — | 50 |
| Evaluation | | | | |
| Dry Oil Formation | D | B | A | D |
| Impact Resistance | D | C | A | D |
| Vibration Stability | D | D | A | D |

Referring to the above Table 1, when sericite without the fluorine treatment was used as the oil-enclosable powder, it was difficult to produce an oil-enclosed powdery composition. However, when fluorine-treated sericite was used, an oil-enclosed powdery composition could be prepared.

However, even when fluorine-treated sericite was used, the adjustment of shares was difficult during the production of compositions and the composition turned to paste by impact following the preparation of the oil-enclosed powdery composition, in case.

On the other hand, when fluorine-treated aluminum-hydroxide-coated sericite was used, excellent production applicability, impact resistance and vibration stability could be achieved, but even in the case of the same fluorine-treated aluminum-hydroxide-coated sericite, one time the effect was high but another time the effect was poor, and further, the correlation between the oil and the effect was significant.

Following, the present inventors have further investigated fluorine-treated metal oxide/hydroxide coated powder. The results are shown in Table 2 below.

TABLE 2

| | | | Maximum Amount of | | |
|---|---|---|---|---|---|
| | Contact | | Enclosed Oil | | |
| | Angle | | Liquid | | Dry oil |
| | Water | Oil | paraffin | Squalane | formation |
| Fluorine-treated | 135 | 133 | 2.54 | 2.45 | B |
| Alminum-hydroxide- | | | | | |
| coated (7.5%) | | | | | |
| sericite | | | | | |
| Fluorine-treated | 145 | 142 | 3.135 | 2.865 | A |
| Aluminum-hydroxide- | | | | | |
| coated (10%) | | | | | |
| sericite | | | | | |
| Fluorine-treated | 135 | 131 | 2.103 | 1.66 | C |
| Barium-hydroxide- | | | | | |
| coated sericite | | | | | |
| Fluorine-treated | 132 | 130 | 1.963 | 1.708 | C |
| Calcium-hydroxide- | | | | | |
| coated sericite | | | | | |
| Fluorine-treated | 152 | 148 | 2.105 | 1.755 | C |
| Titanium-hydroxide- | | | | | |
| coated sericite | | | | | |
| Fluorine-treated | — | — | — | — | D |
| zinc-oxide-coated | | | | | |
| sericite | | | | | |
| Fluorine-treated | — | — | — | — | D |
| bismuth | | | | | |
| oxychloride- | | | | | |
| coated sericite | | | | | |

Zinc oxide and bismuth could not coat metal.

Referring to Table 2, even though it is clear that the fluorine-treated titanium-hydroxide-coated sericite provides a high contact angle against both water and oil, the fluorine-treated aluminum-hydroxide-coated sericite gives the higher maximum amount of enclosed oil as a trend and accordingly, it is understandable that the high amount of enclosed oil relative to the fluorine-treated aluminum-hydroxide-coated sericite is specific.

Following, the present inventors investigated the condition of aluminum hydroxide coating. Specifically, the process of coating sericite with aluminum hydroxide was carried out as described below, wherein the precipitation time varied sequentially. As a result, the particle size (specific surface area) of the precipitation was variable while the coating amount of aluminum hydroxide is being kept nearly constant.

### Precipitation Method of Aluminum Hydroxide

Short precipitation time=If the dropping speed of acid is fast, fine aluminum hydroxide particles are formed=providing a large specific surface area.
Long precipitation time=If the acid is slowly dropped, large particles of aluminum hydroxide are formed=providing a small specific surface area.

TABLE 3

| | | | Maximum | | |
|---|---|---|---|---|---|
| | | Specific | Amount of | | |
| | Precipitation | Surface | Enclosed | Dry Oil | |
| | time | Area | Oil | Formation | Stability |
| Sericite | — | 5 | 0 | D | — |
| 15% | 3 min | 18.7 | 4 | A | A |
| Aluminum | | | | | |
| Hydroxide | | | | | |
| Coating | | | | | |
| 15% | 10 min | 28.2 | 3.8 | A | A |
| Aluminum | | | | | |
| Hydroxide | | | | | |
| Coating | | | | | |
| 15% | 30 min | 8.3 | 2.9 | C | D |
| Aluminum | | | | | |
| Hydroxide | | | | | |
| Coating | | | | | |
| 15% | 60 min | 8.6 | 2.8 | C | D |
| Aluminum | | | | | |
| Hydroxide | | | | | |
| Coating | | | | | |

Referring to Table 3, it is clear that even when the powder is coated with the same amount of aluminum hydroxide, the specific surface area varies depending on the precipitation time, and accordingly, it is understandable that the production and stability of the oil-enclosed powdery composition are significantly affected.

As a result of the detailed investigation by the present inventors, it was found that the preferable specific surface area was not smaller than 10 m²/g and more preferably 15 to 40 m²/g at the stage as-is aluminum-hydroxide-coated platy mineral before fluorine treatment.

In addition, the present inventors have investigated the relationship between the oil-enclosable powder of the present invention and the surface tension value of the oil. Specifically, liquid paraffin and dimethyl silicone were mixed to adjust the surface tension value, and the production and stability of the oil-enclosed powdery composition were evaluated. The results are shown in Table 4.

TABLE 4

| | Test Examples | | | |
|---|---|---|---|---|
| | 4-1 | 4-2 | 4-3 | 4-4 |
| Fluorine-treated Aluminum hydroxide-coated | 50 | 50 | 50 | 50 |
| Sericite | | | | |
| Liquid Paraffin | 50 | 40 | 30 | 20 |
| Dimethyl Silicone | 0 | 10 | 20 | 30 |
| Surface Tension Value (mN/m) | 30 | 25 | 23 | 19 |
| Dry Oil Formation | A | A | B | C |
| Stability (Drop/Vibration) | A | A | B | D |

Referring to Table 4, it is clear that if the surface tension value is not smaller than 2×10⁻² N/m, both the dry oil formation and the stability are superior as a trend.

Furthermore, the kinds of the oil and the production of the oil-enclosed powdery compositions were investigated. The results are shown in Table 5 below.

TABLE 5

| | Test Examples | | | |
|---|---|---|---|---|
| | 5-1 | 5-2 | 5-3 | 5-4 |
| Fluorine-treated Aluminum hydroxide-coated | 50 | 50 | 50 | 50 |
| Sericite | | | | |
| Olefin Oligomer | 50 | — | — | — |
| Squalane | — | 50 | — | — |
| CIO | — | — | 50 | — |
| Dimethyl Silicone | — | — | — | 50 |
| Surface Tension Value (mN/m) | 29 | 28 | 30 | 17 |
| Dry Oil Formation | A | A | B | D |
| Stability (Drop/Vibration) | A | A | C | D |

Referring to Table 5, it is clear and understandable that the ideal oil especially in the production and stability is hydrocarbon oil.

Thus, the present inventors investigated the cases when the hydrocarbon oil was substituted with other kinds of oil. The results are shown in Table 6.

TABLE 6

| | Test Examples | | | | |
|---|---|---|---|---|---|
| | 6-1 | 6-2 | 6-3 | 6-4 | 6-5 |
| Fluorine-treated Aluminum hydroxide-coated | 50 | 50 | 50 | 50 | 50 |
| Sericite | | | | | |
| Liquid Paraffin | 40 | 30 | 25 | 20 | 10 |
| CIO | 10 | 20 | 25 | 30 | 40 |
| Dry Oil Formation | A | A | A | B | C |
| Stability (Drop/Vibration) | A | A | B | C | C |

Referring to Table 6, it is understandable that satisfactory properties can be provided with practical uses if the amount of hydrocarbon oil based on the oil component is not less than 50% by mass.

Furthermore, the production and stability based on the size of the enclosed oil droplet was evaluated.

Specifically, oil-enclosed powdery compositions were prepared by spraying oil droplets with the specified size while stirring the oil-enclosable powder having the average particle size of 8 to 10 μm. The results are shown in Table 7.

TABLE 7

| | Test Examples | | | | |
|---|---|---|---|---|---|
| | 7-1 | 7-2 | 7-3 | 7-4 | 7-5 |
| Fluorine-treated Aluminum | 50 | 50 | 50 | 50 | 50 |
| hydroxide-coated Sericite | | | | | |
| Liquid Paraffin | 50 | 50 | 50 | 50 | 50 |
| Average Oil Droplet Size | 30 | 40 | 75 | Not | 15 |
| During Spraying (μm) | | | | smaller | |
| | | | | than 100 | |
| Oil-Enclosed Powder Size | 30-100 | 40-100 | 50-120 | 80-150 | — |
| in Product (μm) | | | | | |
| Dry Oil Formation | A | A | A | B | D |
| Stability (Drop/Vibration) | A | A | B | C | D |

Based on the above results, it is understandable that the dry oil that is prepared from oil droplets having the average size of 20 to 80 μm under spraying is stable. Although the accurate measurement of the oil droplet size is difficult in the product, the particle size distribution is preferably 10 to 150 μm and more preferably 10 to 120 μm observed under the microscope.

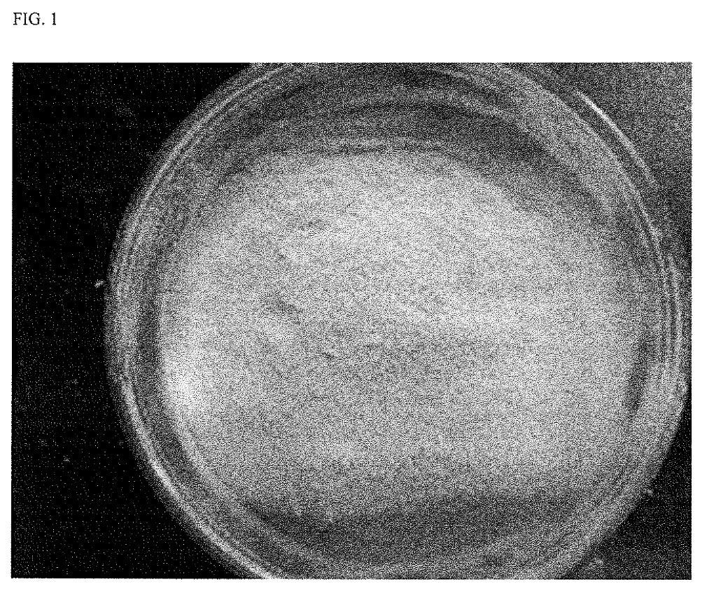

FIGS. 1 to 4 show the results as for the vibration stability test when the average size of the oil droplets was adjusted under spraying.

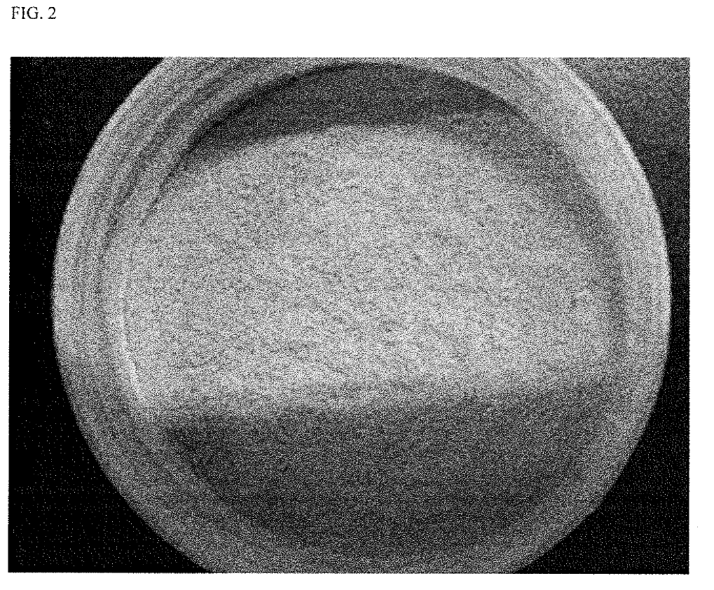

FIG. 1 shows the condition of the oil-enclosed powdery composition that was prepared from oil droplets having the average size of 40 μm under spraying. FIG. 2 shows the condition of the oil-enclosed powdery composition subjected to the vibration stability test. By comparing both FIGs., no specific change was observed as to the condition of the oil-enclosed powdery composition.

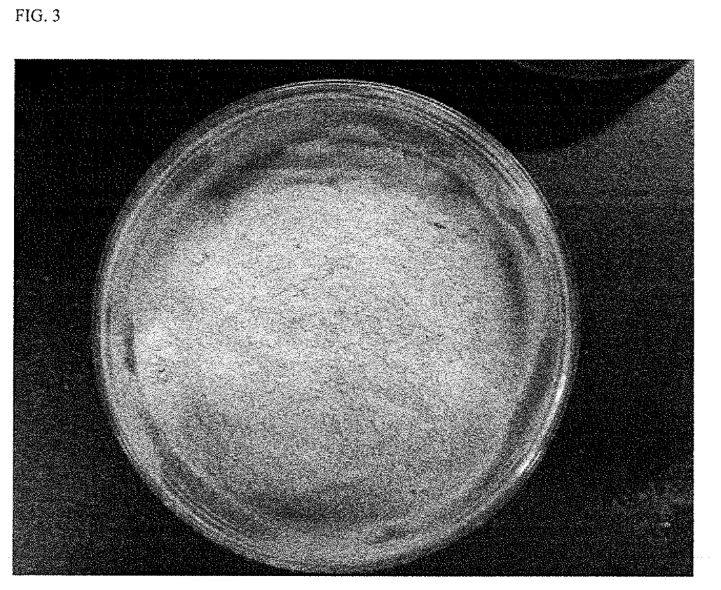

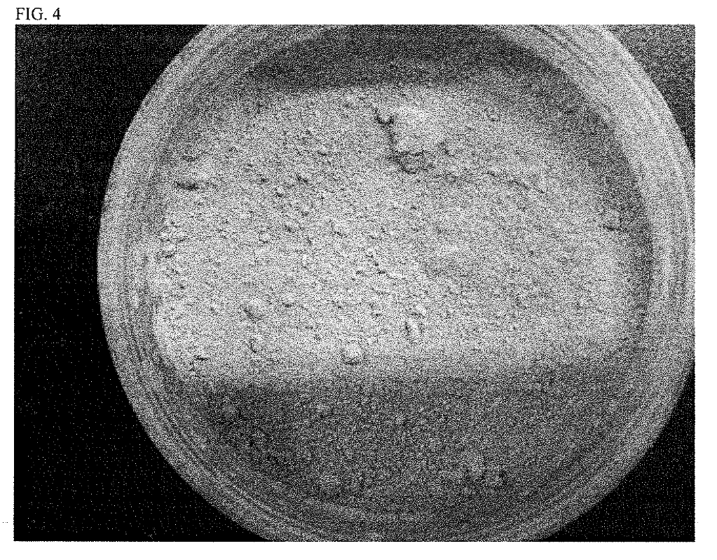

On the other hand, FIG. 3 shows the condition of the oil-enclosed powdery composition that was prepared with oil droplets having the average size of not smaller than 100 μm under spraying. FIG. 4 shows the condition of the oil-enclosed powdery composition subjected to the vibration stability test. Based on FIG. 3, it is understandable that the oil-enclosed powdery composition can be prepared, but parts of the oil-enclosed powder coalesce by vibration.

According to the detailed investigation by the present inventors, when the average size of oil droplets under spraying was 20 to 80 μm, an ideal dry oil could be prepared and the stability thereof under vibration could be provided.

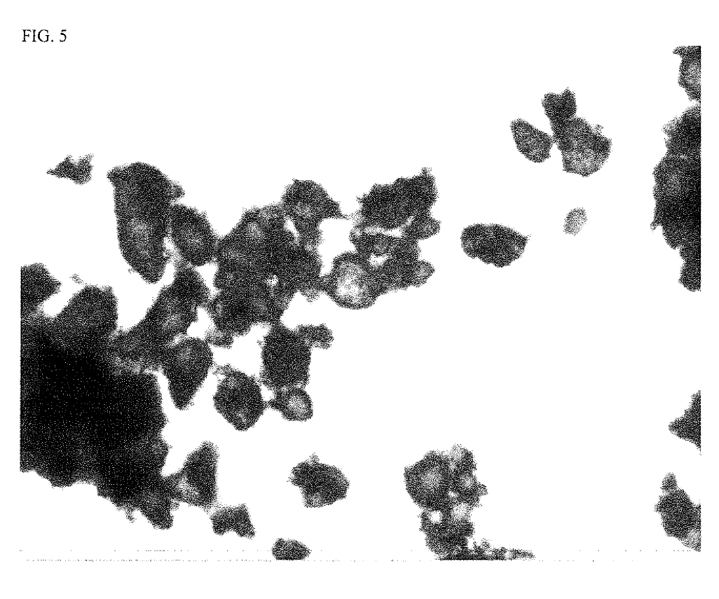

FIG. 5 shows a micrograph of the oil-enclosed powder of the present invention. By the observation of the micrograph, it was clarified that the particle size distribution in the product was 10 to 150 μm and it is preferably 10 to 120 μm.

The oil-enclosed powdery composition and the production method of the present invention are explained based on the above-described aspect of the embodiment and examples. However, the invention is not limited to the above-described aspect of the embodiment and examples. Within the scope of the present invention and based on the basic technological thought of the present invention, various transformations, modifications and improvements for the respective disclosed elements (inclusive of elements described in the claims, description, and drawings) can be included. In addition, various combinations, substitutions and selections of the respective disclosed elements are possible within the scope of the claims of the present invention.

Additional problems, objects, and modes (inclusive of modified modes) of the present invention can also be clarified from the full disclosure of the present invention including the claims.

For the numerical ranges described in the present document, even when there is no specific description, it should be interpreted that arbitrary numerical values and ranges that are within the ranges are specifically described in the present document.

What is claimed is:

1. An oil-enclosed powdery composition, comprising:
an oil-enclosable powder; further comprising;
a platy powder,
aluminum hydroxide coating said platy powder, having a surface of a specific surface area of at least 10 m²/g, and
a fluorine compound coating said aluminum hydroxide; and
an oil droplet having at least 50% by mass of hydrocarbon oil based on a total oil component and at least 2.0×10⁻² N/m of a surface tension value as a total oil component; wherein said oil-enclosable powder encompassing said oil droplet is like powder having an average particle size of 10 to 150 μm and said oil-enclosed powdery composition is liquefiable by inunction.

2. The oil-enclosed powdery composition according to claim 1, wherein the platy powder comprises sericite.

3. The oil-enclosed powdery composition according to claim 1, wherein the ratio of the oil-enclosing powder vs. the oil component is 1:9 to 4:1 (mass ratio).

4. A production method of an oil-enclosed powdery composition, comprising the steps of:
providing an oil-enclosable powder comprising a platy powder; aluminum hydroxide coating said platy powder, having a surface of a specific surface area of at least 10 m²/g; and a fluorine compound coating said aluminum hydroxide;
stirring said oil-enclosable powder while spraying with a spray containing oil droplets having at least 50% by mass of hydrocarbon oil based on a total oil component and at least 2.0×10⁻² N/m of a surface tension value as a total oil component; and
encompassing said oil droplets with said oil-enclosable powder during the step of stirring.

5. The oil-enclosed powdery composition according to claim 2, wherein the ratio of said oil-enclosing powder vs. said oil component is 1:9 to 4:1 (mass ratio).

6. An oil-enclosable powdery composition according to claim 1, wherein said oil component has the total surface tension value of at least 2.1×10⁻² N/m.

7. The oil-enclosed powdery composition according to claim 1, wherein said fluorine compound comprises at least one of perfluoroalkyl phosphoric acid ester diethanolamine salt represented by the following formula (I): 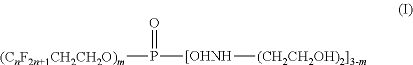
wherein n is an integer of 5 to 20; and m is 1 or 2.

8. The oil-enclosed powdery composition according to claim 1, wherein the fluorine compound comprises at least one of perfluoroalkylsilane represented by the following formula (II):
CₐF₂ₐ₊₁—(CH₂)_{b}—SiX₃ (II)
wherein a is an integer of 1 to 12; b is an integer of 1 to 5; and X is a halogen atom, alkyl group or alkoxy group.

9. The oil-enclosed powdery composition according to claim 1, wherein the fluorine compound comprises at least one of perfluoroalkylethyl acrylate represented by the following (III): 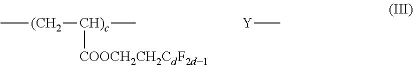
wherein c is an integer of 1 to 12; d is an integer of 5 to 20; and Y is polyethylene glycol, a silicone chain and an alkyl copolymer containing acryl groups.

10. The oil-enclosed powdery composition according to claim 1, wherein
the quantity of the oil-enclosable powder is 10 to 80% by mass based on the total composition.

11. The oil-enclosed powdery composition according to claim 1, wherein
the quantity of the oil component is 20 to 90% by mass.

12. The oil-enclosed powdery composition according to claim 1, wherein
the oil component comprises at least one selected from the group consisting of liquid paraffin, ozokerite, squalene, pristane, paraffin, ceresin, squalane and petrolatum.

13. The production method according to claim 4, wherein the platy powder further comprises sericite.

14. The production method according to claim 4, wherein the ratio of the oil-enclosing powder vs. the oil component is 1:9 to 4:1 (mass ratio).

15. The production method according to claim 4, wherein the oil component has the total surface tension value of at least 2.1×10⁻² N/m.

16. The production method according to claim 4, wherein the fluorine compound comprises at least one of perfluoroalkyl phosphoric acid ester diethanolamine salt represented by the following formula (I): 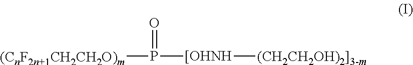
wherein n is an integer of 5 to 20 and m is 1 or 2.

17. The production method according to claim 4, wherein the fluorine compound comprises at least one of perfluoroalkylsilane represented by the following formula (II):
CₐF₂ₐ₊₁—(CH₂)_{b}—SiX₃ (II)
Wherein a is an integer of 1 to 1; b is an integer of 1 to 5; and X is a halogen atom, alkyl group, or alkoxy group.

18. The production method according to claim 4, wherein the fluorine compound comprises at least one of perfluoroalkylethyl acrylate represented by the following (III): 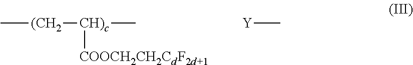
herein c is an integer of 1 to 12; d is an integer of 5 to 20; and Y is polyethylene glycol, a silicone chain, or an alkyl copolymer containing acryl groups.

19. The production method according to claim 4, wherein
the quantity of the oil-enclosable powder is 10 to 80% by mass based on the total composition.

20. The production method according to claim 4, wherein
the quantity of the oil component is 20 to 90% by mass.
